SEQUENCE LISTING (2 sequences)

SEQ ID NO: 1 - DNA, 30 bp, Artificial Sequence
  Primer
atgaaggccttcgagtccctcaagtccttc

SEQ ID NO: 2 - DNA, 28 bp, Artificial Sequence
  Primer
ctcacggtcggtgcagcggctcctcagc

(19) United States
(12) Patent Application Publication
(10) Pub. No.: US 2004/0229837 A1
(43) Pub. Date: Nov. 18, 2004

(54) Treatment of neurodegenerative diseases

(76) Inventors: Yijuang Chern, Taipei (TW); Yi-Chao Lee, Taipei (TW)

Correspondence Address: FISH & RICHARDSON PC, 225 FRANKLIN ST, BOSTON, MA, 02110, US

(21) Appl. No.: 10/440,345

(22) Filed: May 15, 2003

(51) Int. Cl. G01N 33/53; A61K 31/52

(52) U.S. Cl. 514/46; 514/263.4

(57) ABSTRACT

A method of increasing the number of activated astrocytes in a subject. The method involves identifying a subject suffering from or being at risk for developing a neurodegenerative disease and administering to the subject an effective amount of an agonist of the A_{2A}-adenosine receptor. Also disclosed are a method of treating a neurodegenerative disease, a packaged product for treating a subject suffering from or being at risk for developing a neurodegenerative disease, and a method of identifying an agonist of the A_{2A}-adenosine receptor for treating a neurodegenerative disease.

### BACKGROUND

Huntington's disease (HD) is an autosomal dominant neurodegenerative disease caused by a CAG trinucleotide expansion in exon 1 of the Huntingtin (Htt) gene. Htt is widely expressed in brain and many other tissues. Yet, only specific neuronal populations (including enkephalin-positive striatal neurons) are vulnerable to mutant Htt with expanded CAG repeats. The A_{2A}-adenosine receptor (A_{2A}-R), which has been implicated in protection against apoptosis in various cell types, is enriched in Htt/poly(Q)n-sensitive, enkephalin-containing striatal neurons.

### SUMMARY

This invention relates to use of an agonist of the A_{2A}-adenosine receptor in treating a neurodegenerative disease (e.g., Huntington's disease).

In one aspect, the invention features a method of increasing the number of activated astrocytes (i.e., astrocytes that express glial fibrillary acidic protein) in a subject. The method involves identifying a subject suffering from or being at risk for developing a neurodegenerative disease and administering to the subject an effective amount of an agonist of the A_{2A}-adenosine receptor. An agonist of the A_{2A}-adenosine receptor is a compound that increases the expression of the A_{2A}-adenosine receptor gene or the activity of the A_{2A}-adenosine receptor protein. Examples of A_{2A}-adenosine receptor agonists include CGS21680, ATL-146e, ATL-193, and 5N -ethylcarboxamide-adenosine (NECA). The agonist can be administered, e.g., through intraperitoneal injection or intrastriatal injection.

In another aspect, the invention features a method of treating a neurodegenerative disease. The method involves identifying a subject suffering from or being at risk for developing a neurodegenerative disease and administering to the subject an effective amount of CGS21680.

Also within the scope of the invention is a packaged product including a container, an effective amount of CGS21680, and a legend associated with the container and indicating administration of CGS21680 for treating a subject suffering from or being at risk for developing a neurodegenerative disease.

The invention further features a method of identifying an agonist of the A_{2A}-adenosine receptor for treating a neurodegenerative disease. The method involves contacting an astrocyte expressing an A_{2A}-adenosine receptor with an agonist of the A_{2A}-adenosine receptor and determining an activation state of the astrocyte. Activation of the astrocyte in the presence of the agonist indicates that the agonist is a candidate for treating a neurodegenerative disease.

The details of one or more embodiments of the invention are set forth in the accompanying description below. Other features, objects, and advantages of the invention will be apparent from the detailed description, and from the claims.

### DETAILED DESCRIPTION

The present invention is based on an unexpected discovery that an A_{2A}-R selective agonist, CGS21680 (CGS), effectively improved several major pathological characteristics of HD in an HD mouse model (R6/2). As shown in the example below, daily administration of CGS from 7 weeks of age suppressed progressive locomotor deterioration, reversed the increase in striatal choline concentration determined by in vivo proton localized magnetic resonance spectroscopy (¹H-MRS), and reduced brain atrophy in HD mice. Moreover, CGS treatment also markedly enhanced the number of activated astrocytes. Double immunostaining analysis revealed that most of these activated astrocytes contained A_{2A}-R, indicating that CGS activates astrocytes through stimulation of A_{2A}-R. The concurrence of improved symptoms with the increased number of activated astrocytes by CGS treatment in R6/2 mice suggests that activation of astrocytes is beneficial for amelioration of disease progression of HD.

Accordingly, the invention provides a method for treating a neurodegencrative disease with an agonist of the A_{2A}-adenosine receptor and a method for identifying therapeutic compounds for treating such diseases.

An agonist of the A_{2A}-adenosine receptor can be obtained from commercial suppliers or identified according to the methods described below or any other methods well known in the art. Candidate compounds (e.g., proteins, peptides, peptidomimetics, peptoids, antibodies, small molecules or other drugs) can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann et al. (1994) J. Med. Chem. 37:2678-2685; and Lam (1997) Anticancer Drug Des. 12:145.

Examples of methods for the synthesis of molecular libraries can be found in the art, for example, in: DeWitt et al. (1993) PNAS USA 90:6909; Erb et al. (1994) PNAS USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Patent No. 5,223,409), plasmids (Cull et al. (1992) PNAS USA 89:1865-1869), or phages (Scott and Smith (1990) Science 249:386-390; Devlin (1990) Science 249:404-406; Cwirla et al. (1990) PNAS USA 87:6378-6382; Felici (1991) J. Mol. Biol. 222:301-310; and U.S. Pat. No. 5,223,409).

To identify compounds that increase the gene expression or protein activity level of the A_{2A}-adenosine receptor in a system (a cell system or a cell-free system), the system is contacted with a candidate compound and the gene expression or protein activity level of the A_{2A}-adenosine receptor is evaluated relative to that in the absence of the candidate compound. In a cell system, the cell can be a cell that naturally expresses the A_{2A}-adenosine receptor gene, or a cell that is modified to express a recombinant nucleic acid, for example, having the A_{2A}-adenosine receptor gene promoter fused to a marker gene or the coding region of the A_{2A}-adenosine receptor gene fused to a heterologous promoter.

The gene expression level can be determined at either the MRNA level or at the protein level. Methods of measuring mRNA levels in a tissue sample or a body fluid are known in the art. In order to measure mRNA levels, cells can be lysed and the levels of mRNA in the lysates or in RNA purified or semi-purified from the lysates can be determined by any of a variety of methods including, without limitation, hybridization assays using detectably labeled gene-specific DNA or RNA probes and quantitative or semi-quantitative RT-PCR methodologies using appropriate gene-specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantifying mRNA include RNA protection assay (RPA) and SAGE.

Methods of measuring protein levels in a tissue sample or a body fluid are also known in the art. Many such methods employ antibodies (e.g., monoclonal or polyclonal antibodies) that bind specifically to a target protein. In such assays, the antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to bodily fluids or to lysates of cells, and others (e.g., immunohistological methods or fluorescence flow cytometry) applied to histological sections or unlysed cell suspensions. Methods of measuring the amount of label depend on the nature of the label and are well known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

The activity of the A_{2A}-adenosine receptor can be measured, e.g., by radioligand binding assay using ³H-CGS21680 or by measuring the cellular cAMP signaling evoked by stimulation of the receptor (Chern et al. (1993) Mol. Pharm. 44:950-958).

When the gene expression or protein activity level of the A_{2A}-adenosine receptor or the recombinant gene or protein is greater in the presence of the candidate compound than that in the absence of the candidate compound, the candidate compound is identified as an agonist of the A₂A-adenosine receptor.

To identify an agonist of the A2A-adenosine receptor useful for treating a neurodegenerative disease, an astrocyte (in a tissue culture or in an animal model) expressing an A2A-adenosine receptor is contacted with an agonist of the A2A-adenosine receptor, and the activation state of the astrocyte is determined. The activation state of an astrocyte can be determined by detecting expression of glial fibrillary acidic protein (GFAP) in the astrocyte according to the methods described above, e.g., by using Northern blotting, Western blotting, or immunocytochemical staining analysis. If GFAP expression is detected, i.e., the astrocyte is activated, in the presence of the agonist, it indicates that the agonist is a candidate for treating a neurodegenerative disease.

Subjects to be treated for a neurodegenerative disease can be identified, for example, by determining the gene expression or protein activity level of the A2A-adenosine receptor in a sample prepared from a subject by methods described above. If the gene expression or protein activity level of the A_{2A}-adenosine receptor is lower in the sample from the subject than that in a sample from a normal person, the subject is a candidate for treatment with an effective amount of an agonist of the A_{2A}-adenosine receptor.

The term “treating” is defined as administration of a compound to a subject, who has a neurodegenerative disease, with the purpose to cure, alleviate, relieve, remedy, prevent, or ameliorate the disorder, the symptom of the disorder, the disease state secondary to the disorder, or the predisposition toward the disorder. An “effective amount” is an amount of the compound that is capable of producing a medically desirable result, e.g., as described above, in a treated subject.

The treatment method can be performed in vivo or ex vivo, alone or in conjunction with other drugs or therapy.

In one in vivo approach, a therapeutic compound (e.g., a compound that increases the gene expression or protein activity level of the A_{2A}-adenosine receptor) is administered to the subject. Generally, the compound will be suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. For treatment of a neurodegenerative disease, the compound can be delivered directly to the striatum, i.e., through intrastriatal injection.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Wide variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Alternatively, a polynucleotide containing a nucleic acid sequence encoding an agonist of the A_{2A}-adenosine receptor can be delivered to the subject, for example, by the use of polymeric, biodegradable microparticle or microcapsule delivery devices known in the art. Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano, et al. (1995) J. Mol. Med. 73:479). Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the above-mentioned polynucleotides (e.g., expression vectors), the nucleic acid sequence encoding an agonist of the A_{2A}-adenosine receptor is operatively linked to a promoter or enhancer-promoter combination. Enhancers provide expression specificity in terms of time, location, and level. Unlike a promoter, an enhancer can function when located at variable distances from the transcription initiation site, provided a promoter is present. An enhancer can also be located downstream of the transcription initiation site.

Suitable expression vectors include plasmids and viral vectors such as herpes viruses, retroviruses, vaccinia viruses, attenuated vaccinia viruses, canary pox viruses, adenoviruses and adeno-associated viruses, among others.

As is well known in the medical art, the dosage for any one patient depends upon many factors, including the patient's weight, body surface area, age, the particular compound to be administered, sex, time and route of administration, general health, and other drugs being administered concurrently. Dosages will vary, but a preferred dosage for administration of polynucleotide is about 10⁶ to 10¹² copies of the polynucleotide molecule. This dose can be repeatedly administered as needed. Routes of administration can be any of those listed above.

An ex vivo strategy for treating subjects with a neurodegenerative disease can involve transfecting or transducing cells obtained from the subject with a polynucleotide encoding an agonist of the A_{2A}-adenosine receptor. Alternatively, a cell can be transfected in vitro with a vector designed to insert, by homologous recombination, a new, active promoter upstream of the transcription start site of the naturally occurring endogenous A_{2A}-adenosine receptor agonist gene in the cell's genome. Such methods, which “switch on” an otherwise largely silent gene, are well known in the art. After selection and expansion of a cell that expresses the A_{2A}-adenosine receptor agonist at a desired level, the transfected or transduced cells are then returned to the subject. The cells can be any of a wide range of types including, without limitation, neural cells, hemopoietic cells (e.g., bone marrow cells, macrophages, monocytes, dendritic cells, T cells, or B cells), fibroblasts, epithelial cells, endothelial cells, keratinocytes, or muscle cells. Such cells act as a source of the A_{2A}-adenosine receptor agonist for as long as they survive in the subject.

The ex vivo methods include the steps of harvesting cells from a subject, culturing the cells, transducing them with an expression vector, and maintaining the cells under conditions suitable for expression of the agonist of the A_{2A}-adenosine receptor. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the A_{2A}-adenosine receptor agonist. The cells may then be injected or implanted into the subject.

Also within the scope of the invention is a packaged product including a container, an effective amount of an agonist of the A_{2A}-adenosine receptor, and a legend associated with the container and indicating administration of the agonist for treating a subject suffering from or being at risk for developing a neurodegenerative disease. The agonist can be admixed with a pharmaceutically acceptable carrier, including a solvent, a dispersion medium, a coating, an antibacterial and antifungal agent, and an isotonic and absorption delaying agent.

The agonist can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the ligand with a solid carrier and a lubricant. Examples of solid carriers include starch and sugar bentonite. The compound can also be administered in a form of a hard shell tablet or a capsule containing a binder, e.g., lactose or mannitol, a conventional filler, and a tableting agent. The agonist can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent. Further, the agonist can be injected directly to the striatum via brain operation.

The efficacy of the agonist can be evaluated both in vitro and in vivo. For example, the agonist can be tested for its ability to increase gene expression or protein activity of the A_{2A}-adenosine receptor in vitro. For in vivo studies, the agonist can be injected into an animal (e.g., an animal model) and its effects on a neurodegenerative disease are then accessed. Based on the results, an appropriate dosage range and administration route can be determined.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Materials and Methods

Materials

CGS21680 was obtained from Research Biochemicals (Natick, Mass., USA). ZM241385 was purchased from Tocris Cookson Inc. (Ellisville, Mo., USA).

Animals and Drug Administration

Male R6/2 mice and littermate controls were originally obtained from Jackson Laboratories (Bar Harbor, Me., USA), and mated to female control mice (B6CBAFI/J). Offspring were identified by PCR genotyping of genomic DNA extracted from tail tissues using primers located in the transgene (5′-ATGAAGGCCTTCGAGTCCCTCAAGTCCTTC-3′, 5′-CTCACGGTCGGTGCAGCGGCTCCTCAGC-3′) to ensure that the length of the CAG repeat remained approximately 150 (Hogan et al. (1994) In: Manipulating the mouse embryo: a laboratory manual, Ed 2. Cold Spring Harbor, N.Y., Cold Spring Harbor Laboratory). Animals were housed at the Institute of Biomedical Sciences Animal Care Facility under a 12-h light/dark cycle. Animal experiments were performed in accordance with the National Institutes of Health Guidelines under protocols approved by the Institutional Animal Care and Use Committee of IBMS, Academia Sinica.

Adenylyl Cyclase (AC) Assay

AC activity was assayed as described previously (Chem et al. (1993) Mol. Pharmacol. 44:950-958). Briefly, striatal tissues were sonicated and the resultant homogenate was centrifuged at 50,000 ×g for 30 min to collect the P1 membrane fractions. The AC activity assay was performed at 37° C. for 10 min in a 400 ul reaction mixture containing 1 mM ATP, 100 mM NaCl, 50 mM Hepes, 0.2 mM EGTA, 0.5 mM 3-isobutyl-1-methylxanthine, 6 mM MgCl2, 1 uM GTP, and 20 ug of membrane proteins. Reactions were stopped by addition of 0.6 ml 10% TCA. The cAMP formed was isolated by Dowex chromatography (Sigma, St. Louis, Mo., USA) and assayed as described (Chem et al. (1993) Mol. Pharmacol. 44:950-958). The enzyme activity was linear for up to 30 min with up to 40 ug of membrane proteins.

Locomotor Activity

Twenty-four hours after an injection, locomotor activity was measured for 10 min as described (Lee et al. (1992) Chin. J. Physiol. 35:317-336). Briefly, animals were placed in an activity monitor (Coulboum Instrument, Allentown, Pa., USA) equipped with 16 ×16 horizontal sensors. These sensors were used to localize the animal's floor position. Locomotor activity was measured by the total number of beam breaks in an X-Y plane recorded every 10 ms.

In Vivo Proton Localized Magnetic Resonance Spectroscopy (¹H-MRS)

Animals were anesthetized using chloral hydrate (4.088 mg/10 g, i.p. injection). Measurements were performed on a Biospec 4.7 T spectrometer with an active shielding gradient at 6.9 G/cm in 500 us. Mice were placed in a prone position with a custom-designed head-holder. A 20-cm birdcage coil was used for RF excitation, and a 2-cm-diameter surface coil placed directly over the head was used for signal receiving. The volume of interest (VOI) for ¹H-MRS measurements over the striatum was selected on the basis of coronal diffusion-weighted image using a pulse gradient spin-echo diffusion method with a repetition time (TR) of 1500 ms, an echo time (TE) of 62 ms, a field of view of 3 cm×3 cm, a slice thickness of 1 mm, a b value of 1300 s/mm², number averages of 2, a 256 -128 matrix size zero filled to 256 -156. The diffusion-sensitive gradients were applied in the read (×) direction before and after the refocusing pulse. Point-resolved spectroscopy (PRESS) sequence, preceded by three consecutive chemical shift selective saturation (CHESS) pulses for water suppression, was used for localized spectroscopy with a 3.5 ×3.5 ×3.5 mm³voxel located in the striatum region, a spectral width (sw) of 4000 Hz, a TR of 3.5 s, a TE of 136 ms, signal averages of 256, and total scanning time of 8 min 32 sec. The peak areas of NAA, choline (Cho), and creatine (Cr), were recognized. The ratios of striatum metabolites relative to Cr were used for statistical analysis.

Brain Tissue Preparation

Animals were deeply anesthetized with sodium pentobarbital (100 ug/g), and intracardially perfused with 4% paraformaldehyde in 0.1 M phosphate buffer (PB, pH 7.4). Brains were carefully removed, post-fixed with 4% paraformaldehyde/0. 1 M PB for 2-5 h, and then immersed in 30% glycerol in 0.1 M PB. Tissues were cut at 20 um on a freezing microtome (CM3050, Leica Microsystems Nussloch GmbH, Nussloch, Germany).

Immunohistochemistry

Single antigen immunohistochemistry was carried out by the avidin-biotin-peroxidase complex method as previously described (Liu et al. (1998) FEBS Lett. 436:92-98). In brief, free-floating sections were incubated in the polyclonal anti-GFAP antiserum (1 :1000 dilution, Sigma, St. Louis, Mo., USA) at 4° C. for 36-48 h followed by a biotinconjugated goat anti-rabbit IgG (1:500, Vector Laboratories, Burlingame, Calif., USA) for 2 h, and then with an avidin-biotin-peroxidase complex for 2 h with three rinses of 0.1 M PB in between. Sections were incubated in 0.1 M PB containing 0.02% DAB and 0.08% nickel ammonium sulfate. Immunostaining was developed by adding H2O2 to a final concentration of 0.0024%. For double immunofluorescence staining, sections were incubated at 4° C. for 36-48 h in a mixture of rabbit polyclonal anti-A_{2A}-R antibody (1 :1000 dilution) and mouse monoclonal GFAP antibody (1: 1000, Sigma, St. Louis, Mo., USA), followed by incubation in a mixture of secondary antibodies containing 0.1% normal goat serum with 1:500 FITC-conjugated goat anti-rabbit IgG (Cappel™ Research, Durham, N.C., USA) and 1:1000 Alexa 568-conjugated goat antimouse IgG (Molecular Probes Inc, Eugene, Oreg., USA) for 2 h. Sections were rinsed several times with 0.1 M PBS and were mounted on slides with 50% glycerol/0.1 M PB. The pattern of double immunostaining was analyzed with the aid of a laser confocal microscope (Bio-Rad, MRC-1000, Hercules, Calif., USA). Elimination of the primary antibody resulted in loss of immunofluorescence staining.

Stereology and Quantification

Serial-cut coronal tissue sections from the rostral segment of the neostriatum to the level of the anterior commissure (interaural 5.34 mm/bregma 1.54 mm to interaural 3.7 mm/bregma -0.1 mm) were used to define atrophy of the striatum and enlargement of the lateral ventricles. Total areas of the striatum or the lateral ventricles were measured using the software, NIH Image 1.62. Nissl- and GFAP-stained sections were quantified by counting the corresponding types of cells in defined areas of photomicrographs taken using a microscope in phase contrast.

RESULTS

It was previously reported that stimulation of A_{2A}-R protected PC12 cells from apoptosis and rescued the blockage of NGF-induced neurite outgrowth caused by suppression of the MAPK cascade (Huang et al. (2001) J. Biol. Chem. 276:13838-13846, and Cheng et al. (2002) J. Biol. Chem. 277:33930-33942). Since A_{2A}-R is located in striatal GABAergic neurons, which are severely degenerated during progression of HD, it is possible that stimulation of A_{2A}-R produces a neuronal protective effect in HD. Since expression of striatal A_{2A}-R is markedly reduced during the progression of HD, probably due to loss of striatal GABAergic neurons (Ferre et al. (1993) J. Neurosci. 13:5402-5406, Saudou et al. (1998) Cell 95:55-66, and Glass et al. (2000) Neuroscience 97:505-519), whether the striatal A_{2A}-R remains functional in a transgenic HD mouse model (R6/2) was first investigated. R6/2 mice harbor the promoter and the exon 1 of the human Htt gene with 144 CAG repeats, and develop a progressive HD syndrome (e.g., deterioration of motor coordination) at age between 9 and 11 weeks (Mangiarini et al. (1996) Cell 87:493-506). Consistent with previous studies (Luthi-Carter et al. (2002) Hum. Mol. Genet. 11:1927-1937), a significant reduction in the level of striatal A_{2A}-R protein of R6/2 mice at the age of 9 weeks was found. Surprisingly, AC activities activated by an A_{2A}-R-selective agonist (CGS) in wild-type and R6/2 mice were very similar. This finding agrees with the observation reported by Varani and colleagues ((2001) FASEB J. 15:1245-1247), suggesting that the A_{2A}-R's signal is aberrantly amplified in a striatal cell line overexpressing mutant Htt with expanded CAG repeats. In addition, daily injection of CGS for 2 weeks in R6/2 mice did not significantly affect the protein expression level or the activity of A_{2A}-R.

The therapeutic effect of an A_{2A}-R-selective agonist (CGS) in R6/2 mice was examined next. R6/2 mice were daily i.p. administrated CGS (5 ug/g) or vehicle starting at the age of 7 weeks for up to 5 weeks. After 2 weeks of treatment, deterioration of the locomotor activity of R6/2 mice was significantly ameliorated by CGS, whereas mice treated with vehicle exhibited a typical decline in locomotor activity. This ameliorating effect of CGS on locomotor activity in R6/2 mice could be blocked by an A_{2A}-R-selective antagonist (ZM241385, 7.5 ug/g, Palmer et al. (1995) Mol. Pharmacol. 8:970-974), demonstrating that the effect of CGS is mediated by A_{2A}-R. In contrast, motor coordination assessed by rotarod performance of R6/2 mice was not affected by CGS treatment.

To monitor neurochemical changes, R6/2 mice injected with CGS or vehicle were analyzed using ¹H-MRS at the age of 9 weeks. N-acetylaspartate (NAA) is regarded as a neuronal marker. Reduction in the concentration of NAA reflects axonal dysfunction and/or loss. The NAA/creatine ratio of vehicle-treated R6/2 mice was much lower than that of wild-type mice, indicating significant neuronal damage in R6/2 mice. This reduction in the NAA/creatine ratio in R6/2 mice was not affected by chronic CGS treatment (0.67±0.02 and 0.69±0.02, respectively, p=0.691). Instead, CGS administration reversed the elevated choline/creatine ratio in R6/2 mice (1.74±0.12 and 1.44±0.08, respectively, p=0.023). The choline/creatine ratio in R6/2 mice was much higher than that of wild-type mice. Such an increase in choline-containing compounds has also been reported with other traumas (Waters et al. (2002) Biochem. Pharmacol. 64:67-77). Changes in choline levels might influence the composition of choline-containing phospholipids (e.g., lysophosphatidylcholine, phosphatidylcholine) in plasma membranes, and subsequently alter the electrophysiological activity as reported in other cell types (Pu and Masland (1984) J Neurosci. 4:1559-1576, and Shander et al. (1996) J. Mol. Cell. Cardiol. 28:743-753). It is interesting to note that significant changes in a wide variety of electrophysiological properties including depolarized resting membrane potentials, and altered action potentials have been found in R6/2 mice (Klapstein et al. (2001) J. Neurophysiol. 86:2667-2677). Chronic CGS treatment may reverse the altered neuronal characters by reducing the elevated choline content in R6/2 mice to a level similar to that of wild-type mice.

Alternatively, elevated choline content may reflect a change in cell types. Since choline is highly concentrated in glia cells (Urenjak et al. (1993) J. Neurosci. 13:981-989), the enhanced choline/creatine ratio in the brain of vehicle-treated R6/2 mice may result from an increase in the number of glia cells. This hypothesis is of particular interest because gliosis is found in the brain of human HD patients (Lange et al. (1976) J. Neurol. Sci. 28:401-425). The number of glia was determined using the Nissl staining technique. Consistent with previous findings in the brains of HD patients (Lange et al. (1976) J. Neurol. Sci. 28:401-425), an approximately 50% increase in the overall glia number in the cortex of R6/2 mice compared to those of the wild-type mice was observed. In contrast, no statistically significant difference in number of glia cells was observed in the striatum of the wild-type and R6/2 mice. Nevertheless, chronic CGS treatment did not affect the number of glial cells in R6/2 mice. Thus, the increased choline/creatine ratio in the striatum of R6/2 mice, which can be reduced by chronic CGS treatment, was not caused by an alteration in the number of glia. The number of astrocytes, the most numerous of glial cells, was also assessed by staining the brain sections using an antibody against an astrocytic marker (glial fibrillary acidic protein, GFAP). Compared to that in wild-type mice, the number of GFAP-positive cells in the striatum of R6/2 mice was only slightly higher at 12 weeks of age, when dramatic deterioration in the locomotor activity of R6/2 mice was observed. Surprisingly, chronic CGS treatment markedly enhanced the number of activated astrocytes in the striatum and in the cortex by 8-fold. Treating R6/2 mice with an A_{2A}-R-selective antagonist (ZM) and CGS simultaneously reduced the CGS-evoked increased number of activated astrocytes in the striatum to a level (43±8/mm², n =4) lower than that of the vehicle-treated R6/2 brain (68±13/mm²,n=5), suggesting the involvement of A_{2A}-R in the activation of astrocytes in R6/2 mice. Double immunohistochemical analysis demonstrated that the majority of astrocytes in CGS-treated HD mice contained endogenous A_{2A}-R. It is therefore likely that chronic CGS treatment activates astrocytes directly by A_{2A}-R stimulation.

Since the addition of ZM abated both the ameliorating effect on locomotor activity and the increased number of activated astrocytes observed in CGS-treated R6/2 mice, astrocytes may exert protective effects on HD as reported in several disease models (Vila et al. (2001) Curr. Opin. Neurol. 14:483-489). Trophic factors including those secreted by astrocytes (e.g., glial cell line-derived neurotrophic factor, ciliary neurotrophic factor) have been shown to exhibit neuronal protective effects in animal models of HD (Rudge et al. (1992) Eur. J. Neurosci. 4:459-471, Alberch et al. (2002) Brain Res. Bull. 57:817-822, and Mittoux et al. (2002) J. Neurosci. 22:4478-4486). It is possible that the ameliorating effect of chronic CGS treatment is mediated through astrocyte-based trophic support. Alternatively, glial cells may protect neurons by scavenging toxic compounds. For example, glial cells are responsible for effective clearance of glutamate, a major cause of excitotoxicity, through glutamate transporters. Elevation of cAMP levels has been shown to up-regulate the expression of glutamate transporters in primary astrocyte cultures (Gochenauer and Robinson (2001) J. Neurochem. 78:276-286). In the brain of R6/2 mice, the expression and activity of glutamate transporters are reduced, indicating derangement in the handling of glutamate (Lievens et al. (2001) Neurobiol. Dis. 8:807-821, and Behrens et al. (2002) Brain 125:1908-1922). Because activation of A_{2A}-R raises cAMP levels, chronic CGS treatment may enhance the expression of glutamate transporter (Gochenauer and Robinson (2001) J. Neurochem. 78:276-286) and may subsequently attenuate the progression of neurodegeneration by facilitating the removal of synaptic glutamate.

Another major characteristic of HD is striatal atrophy. In R6/2 mice, marked progressive atrophy of the striatum from the ages of 3 to 13 weeks was reported (Ferrante et al. (2000) J. Neurosci. 20:4389-4397). Chronic administration of CGS reduced the size of ventricular enlargement (1.63±0.14 and 091±0.14 mm², for vehicle- and CGS-treated R6/2 mice, respectively, p<0.001, n=5) at 12 weeks of age.

Stimulation of A_{2A}-R has been shown to protect against cell death in many different cell types. In the present study, chronic administration of CGS leads to a protective effect on several important symptoms (e.g., locomotor deterioration, increase in choline-containing compounds, and ventricular enlargement) of HD. Popoli and colleagues ((2002) J. Neurosci. 22:1967-1975) reported that low dosage of an A_{2A}-R antagonist (SCH58261) reduced QA-induced excitotoxicity in rats, which has been used as an excitotoxic rat model of HD. Through an unknown mechanism, blockage of A_{2A}-R appears to modulate the glutamate outflow in the striatum of wild-type rats (Corsi et al. (2000) Neuroreport 11:2591-2595), which might contribute to the beneficial effect of SCH58261 in QA-lesioned animals. However, it should be pointed out that although the QA-lesioned excitotoxic model of HD mimics many of the neuropathological features of HD patients, it is well established that expression of mutant Htt with expanded CAG repeats acquires novel functions and characters which are absent from wild-type animals (gain of function, Rubinsztein (2002) Trends Genet. 18:202-209). For example, R6/2 mice which express mutant Htt are resistant (or less sensitive) to toxicity caused by QA, kainic acid, or a mitochondria toxin (3-nitropropionic acid, 3NP) (Hansson et al. (1999) Proc. Natl. Acad. Sci. USA 96:8727-8732, Hickey and Morton (2000) J. Neurochem. 75:2163-2171, Morton and Leavens (2000) Brain Res. Bull. 52:51-59). Moreover, profound changes in electrophysiological properties and signal transduction of G protein-coupled receptors (e.g., A_{2A}-R) were reported in cells over-expressing mutant Htt (Klapstein et al. (2001) J. Neurophysiol. 86:2667-2677, and Varani et al. (2001) FASEB J. 15:1245-1247). Pharmacological responses in HD models created by lesioning of specific neuronal populations or expression of mutant Htt with expanded CAG repeats can therefore greatly differ. In the present study, chronic treatment with CGS in a transgenic HD mouse model (R6/2) led to significant benefits against the progression of HD in an A_{2A}-R-dependent manner. In contrast, chronic IP-administration of CGS evoked no detectable changes in choline/creatine levels in wild-type mice. The number of activated astrocytes was also not affected by chronic CGS treatment in wild-type mice. It is apparent that stimulation by A_{2A}-R exhibits different physiological responses in R6/2 and wild-type animals. Agonists and antagonists of A_{2A}-R therefore may exhibit distinct beneficial effects in animal models created using different insults.

It was also noticed that some symptoms of HD (e.g., decline in rotarod perfonnance, reductions in NAA concentrations) could not be ameliorated with the current protocol of CGS treatment. The inability of CGS to affect these symptoms may be due to their early onset during HD progression. It has been documented that rotarod performance of R6/2 mice markedly declines from the ages of 4 to 6 weeks. Reduced motor coordination is maintained at a steady level until mice are 11 weeks old (Ferrante et al. (2000) J. Neurosci. 20:4389-4397). Likewise, a significant reduction in striatal NAA levels was observed in R6/2 mice at 4 weeks of age (Jenkins et al. (2000) J. Neurochem. 74:2108-2119). In the present study, chronic CGS treatment of R6/2 mice began at the age of 7 weeks. The lack of a CGS effect on rotarod performance and decreased NAA concentrations may have been due to irreversible neuronal damage which had already occurred before CGS treatment took place. Administration of CGS at an earlier age (e.g., 4 weeks) therefore may further enhance the protective action of CGS observed in the present study, and is likely to provide a useful therapeutic strategy for HD.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of increasing the number of activated astrocytes in a subject, the method comprising:
identifying a subject suffering from or being at risk for developing a neurodegenerative disease, and
administering to the subject an effective amount of an agonist of an A_{2A}-adenosine receptor.

2. The method of claim 1, wherein the neurodegenerative disease is Huntington's disease.

3. The method of claim 2, wherein the agonist is CGS21680.

4. The method of claim 3, wherein the agonist is administered through intraperitoneal injection.

5. The method of claim 3, wherein the agonist is administered through intrastriatal injection.

6. The method of claim 1, wherein the agonist is CGS21680.

7. The method of claim 6, wherein the agonist is administered through intraperitoneal injection.

8. The method of claim 6, wherein the agonist is administered through intrastriatal injection.

9. A method of treating a neurodegenerative disease, the method comprising:
identifying a subject suffering from or being at risk for developing a neurodegenerative disease, and
administering to the subject an effective amount of CGS21680.

10. The method of claim 9, wherein CGS21680 is administered through intraperitoneal injection.

11. The method of claim 9, wherein CGS21680 is administered through intrastriatal injection.

12. The method of claim 9, wherein the neurodegenerative disease is Huntington's disease.

13. The method of claim 12, wherein CGS21680 is administered through intraperitoneal injection.

14. The method of claim 12, wherein CGS21680 is administered through intrastriatal injection.

15. A packaged product comprising:
a container,
an effective amount of CGS21680, and
a legend associated with the container and indicating administration of CGS21680 for treating a subject suffering from or being at risk for developing a neurodegenerative disease.

16. The packaged product of claim 15, wherein the legend indicates that the administration of CGS21680 is through intraperitoneal injection.

17. The packaged product of claim 15, wherein the legend indicates that the administration of CGS21680 is through intrastriatal injection.

18. The packaged product of claim 15, wherein the neurodegenerative disease is Huntington's disease.

19. The packaged product of claim 18, wherein the legend indicates that the administration of CGS21680 is through intraperitoneal injection.

20. The packaged product of claim 18, wherein the legend indicates that the administration of CGS21680 is through intrastriatal injection.

21. A method of identifying an agonist of an A_{2A}-adenosine receptor for treating a neurodegenerative disease, the method comprising:
contacting an astrocyte expressing an A_{2A}-adenosine receptor with an agonist of the A_{2A}-adenosine receptor, and
determining an activation state of the astrocyte, wherein activation of the astrocyte in the presence of the agonist indicates that the agonist is a candidate for treating a neurodegenerative disease.

22. The method of claim 21, wherein the neurodegenerative disease is Huntington's disease.
